(19) United States
(12) Patent Application Publication
(10) Pub. No.: US 2019/0247567 A1
(43) Pub. Date: Aug. 15, 2019

(54) Percutaneous Joint Lavage Devices and Kits

(71) Applicant: Biosurgery Solutions LLC, West Orange, NJ (US)

(72) Inventors: Wilmer L Sibbitt, Jr., Albuquerque, NM (US); Philip Arthur Band, West Orange, NJ (US); Randy Robert Sibbitt, Helena, MT (US)

(21) Appl. No.: 16/273,151

(22) Filed: Feb. 12, 2019

Related U.S. Application Data
(60) Provisional application No. 62/629,677, filed on Feb. 12, 2018.

(51) Int. Cl. A61M 3/02; A61M 1/00

(52) U.S. Cl. CPC A61M 3/0233; A61M 1/0031; A61M 3/0287; A61M 1/0064

(57) ABSTRACT

Percutaneous musculoskeletal lavage devices, and procedures for simultaneous therapeutic and diagnostic purposes, performed with a needle or catheter and various combinations of a syringe, reciprocating syringe, stop-cock, check valves, depth indicator, compressive sleeve or brace, and specialty catheters, provided individually or in a kit. The lavage fluid can be analyzed for factors of medical interest and used to determine personalized treatment, and has intrinsic medical benefit by virtue of clearing the tissues of noxious substances in an enhanced fashion.

### FIELD OF THE INVENTION

The present invention relates to the field of percutaneous musculoskeletal lavage devices, and procedures for simultaneous therapeutic and diagnostic purposes, performed with a needle or catheter and various combinations of a syringe, reciprocating syringe, stop-cock, check valves, depth indicator, compressive sleeve or brace, and specialty catheters, provided individually or in a kit. This invention relates to percutaneous lavage for simultaneous diagnostic and therapeutic purposes, wherein a needle or catheter is inserted through the skin into normal, traumatized, or diseased musculoskeletal tissues, and lavage fluid is injected into the tissue and then aspirated from the tissue using specially designed lavage devices, solutions and volumes, singly or in kits. The lavage catheter device can be inserted by palpation or probe-guidance or image-guided with ultrasound, magnetic resonance imaging, computed tomography, or fluoroscopy amongst other image guidance methods. The lavage procedure is specifically designed to accomplish therapeutic and diagnostic purposes simultaneously. The aspirated lavage fluid can be analyzed for cells, biologically active factors, chemicals, cytokines, crystals, organisms and any biomarker of medical interest. The lavage process can be performed multiple times (cycled) through a single injection site, to improve mixing between the lavage fluid and endogenous tissue fluid, or to gently access hard-to-reach tissue compartments. The extent of lavage needed for individual patients can be determined using a lavage-completion marker that can be measured at the point of care, to ensure that this marker reaches pre-specified values indicating the adequacy of the therapeutic lavage. The lavage fluid collected can subsequently be analyzed quantitatively with correction for the lavage dilution using methods known to those skilled in the art. The pre-specified biomarkers are chosen such that they can inform treatment decisions. The musculoskeletal tissue site where lavage has been performed can subsequently be injected with various solutions and medications prior to removing the needle or catheter, or at some future date in a follow-on procedure, but preferably the treatment used will depend on the results of biomarker analysis in the lavage fluid recovered, and to customize local treatment based on the use of local biomarkers that identify specific disease phenotypes, and thereby identify appropriate therapeutic targets for local musculoskeletal therapy.

### BACKGROUND ART

Pain, inflammation, swelling, and diminished function are common symptoms of musculoskeletal pathologies, and various protocols exist for the treatment of these symptoms. For example, rheumatoid arthritis is a systemic autoimmune disorder treated with systemic corticosteroids, immunosuppressives, biological response modifiers, and intraarticular injections with corticosteroids. Osteoarthritis, despite being a more localized disease, is likewise treated with systemic agents such as acetaminophen and nonsteroidal anti-inflammatory drugs, but is also treated with local intraarticular injections with corticosteroids or hyaluronic acid and derivatives thereof, as well as topical and other therapies. Tendinopathies and back pain are treated by conservative measures and physical therapy, and sometimes with corticosteroid, platelet-rich plasma or anabolic injection. Lavage by multi-port arthroscopy has been suggested as a treatment for some of these conditions, but is not generally considered as efficacious or cost-effective (McAlindon 2014, American Academy of Orthopaedic Surgeons, NICE 2014). Surgery can also have a role in treating these conditions, but is generally reserved for conditions where correction of traumatic or anatomic defects is necessary to preserve joint function. Surgery and total joint arthroplasty are generally considered a treatment of last resort. There are few available diagnostic criteria to determine which patients will respond to particular treatments. Importantly, quantitative biomarkers of local disease activity obtained by lavage of the synovial cavity can be useful to inform joint-specific surgical decisions, by stratifying patients' risk for rapid disease progression, and the probability of responding to a particular non-surgical treatment as in U.S. Pat. No. 8,883,427 to Band.

The most common type of joint lavage is performed in conjunction with arthroscopic surgery, and uses separate portals for irrigation and aspiration. Percutaneous (closed joint) lavage (irrigation) performed without arthroscopy using a needle or cannula has been used since at least since 1987 for the treatment of arthritis (Dawes 1987). Procedure variations include tidal lavage using a single needle where the joint is filled with fluid through a single needle or cannula and then emptied using the same needle, and continuous lavage where two separate catheters are used for irrigation and drainage (Dawes 1987, Edelson 1995, Hilliquin 1996, Kalunian 2000, Meijer 2000, Avral 2005). Closed needle lavage of the joint has been used to treat osteoarthritis, rheumatoid arthritis, septic arthritis, and crystal induced arthritis amongst other conditions (Chang 1993, Ike 1992,1993, Caporali 1994, Bradley 2002, Sornay-Soares 2004). Saline lavage and saline injection may have effects beyond the washing effect on a joint and appear to actually stimulate hyaluronate production (Tulamo 1991, Saari 1992). All of these reports on the therapeutic benefit of lavage for osteoarthritis utilize lavage volumes of at least 100 ml, and generally flush a liter or more of lavage fluid through the joint. Joint lavage is also used in conjunction with other intraarticular treatments, including intraarticular injection of corticosteroids and hyaluronate derivatives amongst other agents, where lavage is performed first and injection of the treatment second (McCain 1989, Brusie 1992, Sato 1997, Ravaud 1999, Smith 2003, Vad 2003, Frias 2004). Intraarticular lavage is also used to obtain fluid samples for routine analysis or biomarkers of disease (Ratcliffe 1993, Petersson 1997, Kraus 2002, Raza 2003). Prior reports of therapeutic lavage have not incorporated biomarker analyses into their therapeutic strategy. Prior reports of lavage conditions for purposes of biomarker collection utilize small volumes, generally less than 15 ml, to minimize discomfort to the patient, and these biomarker collection procedures neither evaluate nor optimize the therapeutic benefit derived from the lavage.

When performing lavage, there has not been a description of the use of a pressure device to assist with needle placement, enhance initial drainage, and then assist with lavage exchanges by applying constant or intermittent pressure.

There are a number of relevant prior art references regarding lavage devices that may be of use for joint lavage especially when included in a kit. U.S. Pat. No. 3,957,052 Topham describes pumping syringes with check valves used to aspirate fluid into a syringe and then expel the fluid through an exit port. U.S. Pat. No. 4,098,276 to Bloom describes a similar syringe that operates in the opposite direction. U.S. Pat. No. 4,314,586 to Folkman 1980 describes a typical stopcock that could be used in a lavage kit. U.S. Pat. No. 4,595,102 to Cianci describes a procedure tray that could contain a number of different components and could be used for medical procedures. U.S. Pat. No. 5,306,237 to Clement describes a double lumen lavage gun. U.S. Pat. No. 5,330,424 to Palmer and U.S. Pat. No. 4,872,866 to Davis described double barrel lavage syringes where one side injects and the other side aspirates that could be used for lavage. U.S. Pat. No. 5,836,907 to Campbell demonstrates a two catheter gastric lavage kit where one tube provides fresh lavage fluid and the other tube provides for drainage of the contaminated lavage fluid. U.S. Pat. No. 5,964,728 to Lln describes an eye lavage system consisting of multiple check valves and syringes. U.S. Pat. No. 6,371,934 B1 to Jackson describes a joint lavage needle with a debrider tip for use with arthroscopy. Although saline or lactated Ringers or other typical fluids are usually used for lavage, U.S. Pat. No. 5,972,909 to Di Napoli describes the use of hyaluronate compounds as a joint irrigant.

There has been one patent, U.S. Pat. No. 6,527,760 to Vad, describing an outpatient joint lavage kit and its method of use which was filed in 2000, including sterile drapes, needles, local anesthesia and related components. Further descriptions of possible lavage components were described in the joint lavage literature (Dawes 1987, McCain 1989, Brusie 1992, Chang 1993, Ike 1992, 1993, Caporali 1994, Edelson 1995, Hilliquin 1996, Sato 1997, Ravaud 1999).

Deficient in the prior art include specific closed joint lavage systems and kits that permit more accurate needle placement, include safety devices, can accommodate ultrasound-directed procedures, minimize needle trauma to tissues, permit both aspiration and injection using a 1-handed single syringe system, and which can be dedicated to one-way intraarticular lavage, tidal lavage, the use of compressive devices to assist in lavage exchanges and catheter placement, continuous one-catheter lavage, continuous two-catheter lavage, small volume lavage, large volume lavage, lavage with viscous solutions, and lavage followed by injection with a therapeutic substance. The prior art does not describe any lavage devices or procedures that simultaneously optimize the conditions for biomarker collection and the therapeutic benefit derived from the lavage procedure itself.

### DESCRIPTION OF INVENTION

Embodiments of the present invention provide percutaneous musculoskeletal lavage devices, and procedures for simultaneous therapeutic and diagnostic purposes, performed with a needle or catheter and various combinations of a syringe, reciprocating syringe, stop-cock, check valves, depth indicator, compressive sleeve or brace, and specialty catheters, provided individually or in a kit. The lavage fluid can be analyzed for factors of medical interest and used to determine personalized treatment, and has intrinsic medical benefit by virtue of clearing the tissues of noxious substances in an enhanced fashion.

This invention relates to percutaneous lavage for simultaneous diagnostic and therapeutic purposes, wherein a needle or catheter is inserted through the skin into normal, traumatized, or diseased musculoskeletal tissues, and lavage fluid is injected into the tissue and then aspirated from the tissue using specially designed lavage devices, solutions and volumes, singly or in kits. The lavage catheter device can be inserted by palpation or probe-guidance or image-guided with ultrasound, magnetic resonance imaging, computed tomography, or fluoroscopy amongst other image guidance methods. The lavage procedure is specifically designed to accomplish therapeutic and diagnostic purposes simultaneously. The aspirated lavage fluid can be analyzed for cells, biologically active factors, chemicals, cytokines, crystals, organisms and any biomarker of medical interest. The lavage process can be performed multiple times (cycled) through a single injection site, to improve mixing between the lavage fluid and endogenous tissue fluid, or to gently access hard-to-reach tissue compartments. The extent of lavage needed for individual patients can be determined using a lavage-completion marker that can be measured at the point of care, to ensure that this marker reaches pre-specified values indicating the adequacy of the therapeutic lavage. The lavage fluid collected can subsequently be analyzed quantitatively with correction for the lavage dilution using methods known to those skilled in the art. The pre-specified biomarkers are chosen such that they can inform treatment decisions. The musculoskeletal tissue site where lavage has been performed can subsequently be injected with various solutions and medications prior to removing the needle or catheter, or at some future date in a follow-on procedure, but preferably the treatment used will depend on the results of biomarker analysis in the lavage fluid recovered, and to customize local treatment based on the use of local biomarkers that identify specific disease phenotypes, and thereby identify appropriate therapeutic targets for local musculoskeletal therapy.

### BRIEF DESCRIPTION OF THE DRAWINGS

FIG. 1A, FIG. 1B, FIG. 1C, FIG. 1D, FIG. 1E, and FIG. 1F together illustrate the steps and devices required for small volume one-way lavage.

FIG. 2A, FIG. 2B, FIG. 2C, FIG. 2D, FIG. 2E, and FIG. 2F together illustrate the steps and devices required for large volume one-way lavage.

FIG. 3A, FIG. 3B, FIG. 3C, FIG. 3D, FIG. 3E, and FIG. 3F together illustrate the basic process of pressure-assisted joint lavage.

FIG. 4 demonstrates one embodiment of a lavage systemic with valves.

FIG. 5 shows a 4-port stopcock embodiment with a syringe.

FIG. 6 shows a 3-port stopcock embodiment with a syringe.

FIG. 7 shows that simple lavage could also be performed with a syringe or a series of syringes without a stopcock or valve.

FIG. 8A and FIG. 8B together depict an embodiment to permit high volume one-way flow into the intraarticular space using a serious of check valves.

FIG. 9A and FIG. 9B together depict an embodiment to permit high volume one-way flow out of the intraarticular space using a series of check valves.

FIG. 10 demonstrates a preferred embodiment with tubing so that the needle or catheter does not move with manipulations.

FIG. 11 is an alternative embodiment with tubing.

FIG. 12 shows alternative means to fix the lavage needle to the skin.

FIG. 13 demonstrates a two-catheter lavage system.

FIG. 14 shows several possible embodiments for needle and catheters for the lavage systems.

FIG. 15 shows the elements of using a multiport valve for lavage followed by injection of a therapeutic substance.

FIG. 16 demonstrates the basic elements for sub synovial membrane lavage.

### MODES OF CARRYING OUT THE INVENTION AND INDUSTRIAL APPLICABILITY

Devices and kits are disclosed that enable safe, practical and reproducible office-based lavage procedures with combined diagnostic and therapeutic purposes. In all the following systems that joint anatomy and intended needle target can be determined by palpation or with a needle guide, or by the use of direct image visualization with ultrasound, computed tomography, fluoroscopy, or magnetic resonance imaging. The skin will then be cleaned with antiseptic solution, gels, pads, or other such medications and devices. Sterile drapes can be employed to provide a wide sterile field, or a local sterile field without drapes can be used. Local anesthesia can be used, and can consist of topical anesthetics, cooling agents, or injectable local anesthetics. For injectable local anesthetics, a dedicated anesthesia needle and syringe can be used, or the intraarticular introducer needle and syringe can be used to inject local anesthesia. There is the optional use of a pressure device to assist with needle placement, enhance initial drainage, and then assist with lavage exchanges by applying constant or intermittent pressure.

After the procedure, in most cases, pressure can be applied to the puncture site(s), and a sterile bandage strip applied. Consequently, all kits can contain sterile drapes, a vial or bottle of local anesthetic, sterile pads or sponges, a needle to aspirate local anesthetic, a needle to administer the local anesthetic, syringe for the local anesthetic, a syringe for arthrocentesis and needle introduction, a therapy or introducer needle, antiseptic devices and solutions, a syringe or device to inject and/or aspirate lavage fluid, a sterile bandage strip, and potentially lavage solution or other standard kit components. Although conventional syringes and needles, and conventional antiseptics can be used in these kits and are anticipated, in some embodiments to enhance operator safety, anti-needlestick safety needles and syringes can be included, chlorhexidine solutions can be provided instead of povidone, and to enhance patient safety, better-controlled syringes for aspiration and injection, including reciprocating syringes or automatic syringes can be used (U.S. Pat. No. 6,245,046 to Sibbitt, US Application 12899543-2010 to Sibbitt). A procedure tray similar to that described by U.S. Pat. No. 4,595,102 to Cianci can also be used.

Example 1 One-Way Small Lavage Systems with External Compression

In the lavage procedure, the joint is filled with lavage fluid and then drained, usually in a procedure called tidal lavage. US Patent to U.S. Pat. No. 6,527,760 to Vad describes a kit and method for joint lavage. The present invention also describes a lavage system with unique characteristics that can be performed conventionally similar to Vad, but also with the use of an external compression brace. US Patent to U.S. Pat. No. 6,527,760 to Vad does not disclose the use of a compressive device in this procedure to assist in needle placement, arthrocentesis, fluid exchanges, tidal lavage or joint therapy. The use of a compression device to accelerate the exchanges and the completeness of each lavage cycle has not been described, and the present invention fulfills this need.

U.S. Pat. No. 7,468,048 to Meehan 2008 discloses a compressive device for joint aspiration whereby the pressure is provided by integrated pneumatic bladders that fill reversibly with air. The use of this device was implied for arthrocentesis (joint aspiration), but was not envisioned or claimed for lavage and tidal lavage. However, this device would function well for the needle introduction and exchange phases of joint lavage. Similarly the US Provisional Patent to Band 2016 describes an elastomeric sleeve or brace that is used to enhance arthrocentesis, that could also be used in the needle introduction and aspiration cycles of a lavage system. These compressive devices can be used to facilitate the lavage procedure and can be part of a lavage kit according to the present invention.

The device is released as fluid cycles into the joint at low pressure, and the device is engaged as fluid cycles out of the joint at high pressure that is advantageous in this phase of lavage. The device can be made in various sizes to accommodate joint sizes encountered with adults, children, or obese patients, but is most functional in a one-size-fits all design for the knee.

These one-way lavage systems rely first on complete aspiration of any synovial fluid if present for decompression and obtaining samples for biomarkers, and then injection of lavage fluid in a one-way direction. With one way joint lavage, a lavage solution is introduced into the joint via one needle, but the lavage solution is not aspirated out and is left in the joint and relies on the body's fluid homeostasis to absorb the fluid. This type of irrigation differs from primary injection with a therapeutic substance in that after aspiration of all accessible fluid by external compression, the knee is injected with lavage fluid which is usually saline, buffered saline, or other electrolyte solutions. One-way lavage is meant to stimulate local production of hyaluronate and other beneficial factors. One-way lavage employs physiologic saline, Ringers solution, or other physiologically compatible and absorbable or therapeutic fluids.

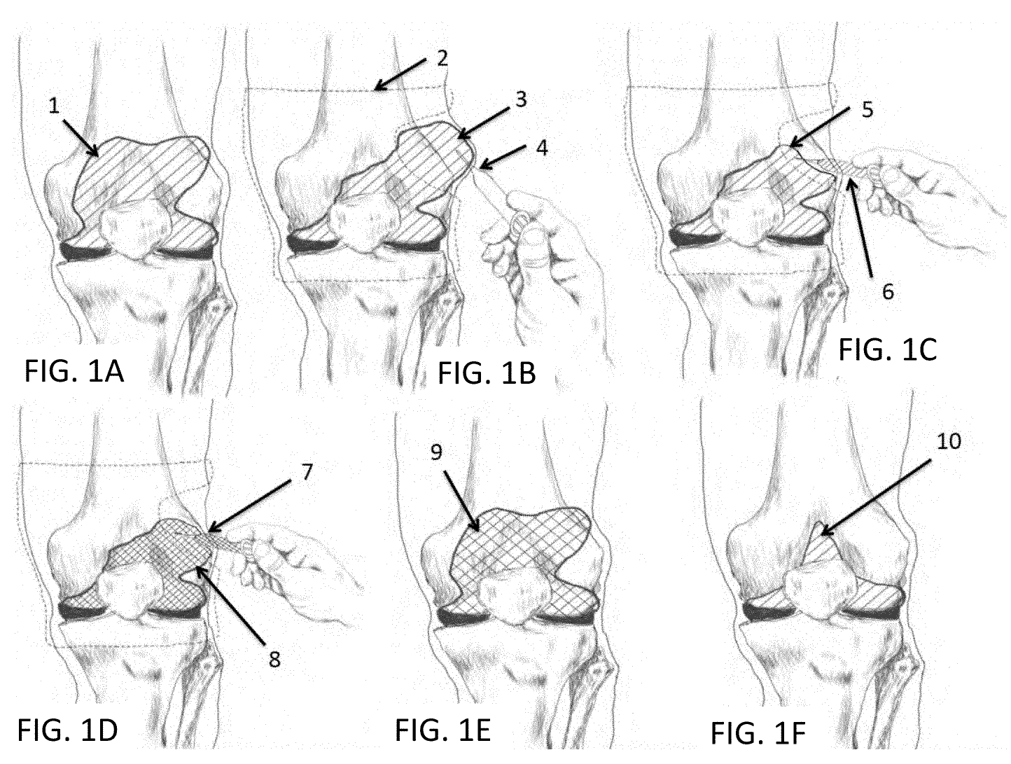

FIG. 1A, FIG. 1B, FIG. 1C, FIG. 1D, FIG. 1E, and FIG. 1F together illustrate the steps and devices required for small volume one-way lavage. FIG. 1A shows a knee with or without intrinsic fluid or an effusion 1 (crosshatch area). The first step in one-way lavage is show in FIG. 1B and comprises placing a compressive brace 2 (broken line) over the knee, the synovial fluid shifts to a more accessible low pressure area 3 in the brace, and a needle or catheter 4 is introduced in the synovial fluid 3. FIG. 1C shows fluid (line hatch) 5 being aspirated into the syringe 6 (line hatch) decreasing the effusion 5 and obtaining synovial fluid for analysis and biomarker assays prior to injecting the lavage fluid. A reciprocating syringe as described U.S. Pat. No. 6,245,046 to Sibbitt or an automatic syringe as in US Application 12899543-2010 to Sibbitt can be advantageous for this procedure, a conventional syringe or a refilling syringe (a syringe with an aspiration spring on the plunger), or other syringe can also function. Needle introduction and complete aspiration of the joint has been shown to be most completely and most reproducibly accomplished with external mechanical compression as described by Bhaysar 2018, Yaqub 2018, and Meehan 2015. Typically a compression brace 2 that permits needle access can be pneumatic as described by U.S. Pat. No. 7,468,048 to Meehan 2008 or mechanical as described by US Patent application to Band 2017 can be placed on the target joint as shown in FIG. 1B. After the synovial fluid is fully aspirated as shown in FIG. 1C, the syringe with fluid is detached from the intraarticular needle, and the lavage syringe with lavage fluid 7 (crosshatch) attached as shown in FIG. 1D. The lavage fluid 7 is then injected from the syringe into the synovial space that fills with lavage fluid 8 (crosshatch). It is anticipated that this type of small volume lavage would be limited by the size of the syringe and would typically vary between 2 ml and 60 ml. The compressive brace (broken line) can be removed after the aspiration phase, or can remain in place during the infusion of lavage fluid as shown in FIG. 1D and then be removed. Other syringe sizes can also be employed. After one-way lavage, the needle can be left in place and the joint can be then be injected with corticosteroid, hyaluronan, or other therapeutic substance through the same needle or catheter. After injection of the lavage fluid (followed by the optional therapeutic substance) the needle is then removed and pressure applied to the puncture site. FIG. 1E demonstrates the lavage fluid 9 (crosshatch) distending the joint, and FIG. 1F shows that with time, the lavage fluid has been completely absorbed collapsing the synovial space onto the articular cartilage 10 (line hatch).

Example 2 Large Volume One-Way Lavage

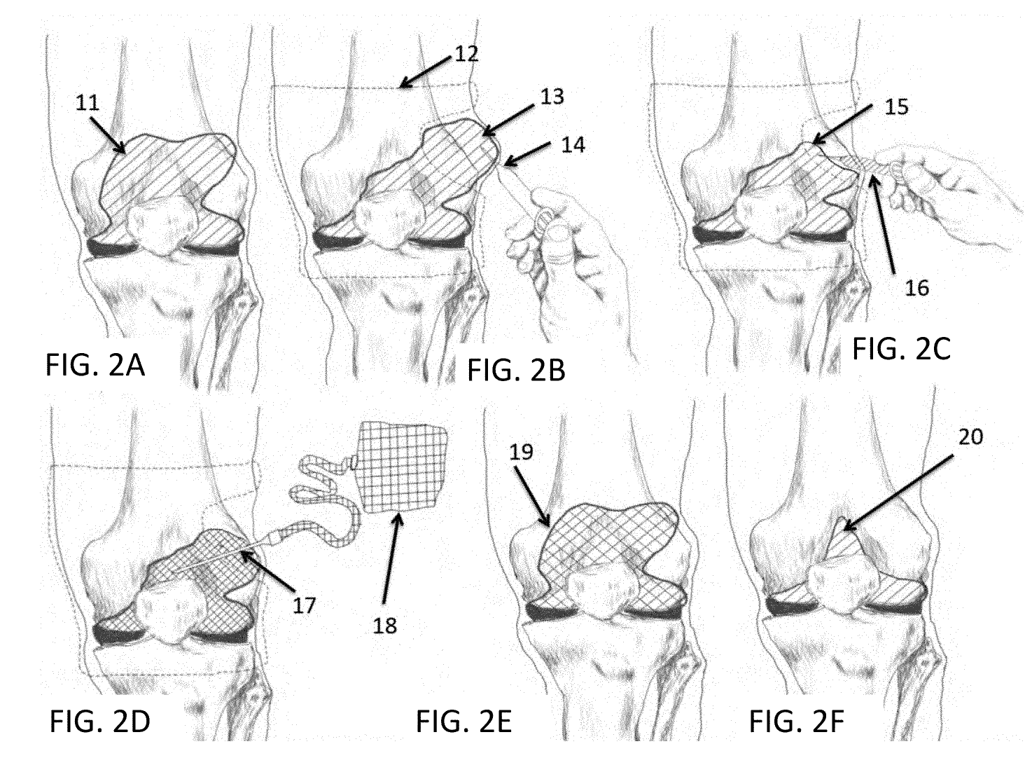

FIG. 2A, FIG. 2B, FIG. 2C, FIG. 2D, FIG. 2E, and FIG. 2F together illustrate the steps and devices required for large volume one-way lavage. Large volume one-way lavage is used to temporarily immobilize the joint, hydro-dissect and hydro-dilate the joint, to facilitate the use of hydraulic forces to gently separate (break up) adhesions, stretch the joint capsule and help lift areas of the capsule pathologically adherent to periosteum, and stimulate the local production of beneficial cytokines, endogenous analgesic factors, and growth factors. Large volume one-way lavage by one definition is greater than 60 ml, and can be accomplished with multiple syringes and sequential syringe exchanges using the same technique as in FIG. 1, but can also be accomplished using a spring-driven syringe, or an intravenous bottle or bag or other pressurized source of fluid. FIG. 2A is a knee with intrinsic synovial fluid 11 (diagonal hatch). As is shown in FIG. 2B, an external compression brace 12 (broken line) is placed on the knee, and the synovial fluid 13 is shifted towards the access point and the introducer needle or catheter 14 is introduced into joint space. FIG. 2C demonstrates synovial fluid 15 (diagonal hatch) is aspirated into the syringe 16 (diagonal hatch) and sent for analysis and biomarker evaluation. As shown in FIG. 2D, the needle or catheter 17 remains in the joint, but instead of attaching a smaller syringe with irrigation fluid to the joint, tubing is attached to the intraarticular needle through a compatible fitting, and the tubing is then attached to a fluid source such as a bag 18 or bottle filled with lavage fluid (cross hatch) through a typical conventional fitting, and the fluid is gravity-fed by using a standard intravenous therapy pole. A disadvantage of this system is that it is low pressure and passive, and can require considerable time for the entire volume of fluid to enter the joint. An automatic syringe (typically spring driven), a pump, or a pressure bottle can speed up this fluid delivery and are contemplated in the present invention. The fluid pressure can be controlled to accommodate various joint characteristics (e.g., small and large), as well as individual patient needs and the extent of pathological adhesions. The compressive brace (broken line) can be removed or loosened to achieve lower intraarticular pressure to permit the joint to expand facilely with lavage fluid. After large-volume one-way lavage is completed, the joint can be injected with corticosteroid, hyaluronan, or other therapeutic substance through the same needle and pressure applied to the joint. FIG. 2E shows the joint immediately after large volume one-way lavage with the lavage fluid 19 (cross hatch) expanding the joint space. FIG. 2F shows the lavage fluid completely absorbed leaving the native joint space and cartilage 20 (diagonal hatch).

Example 3—Compression Assisted Two-Way or Tidal Lavage Systems

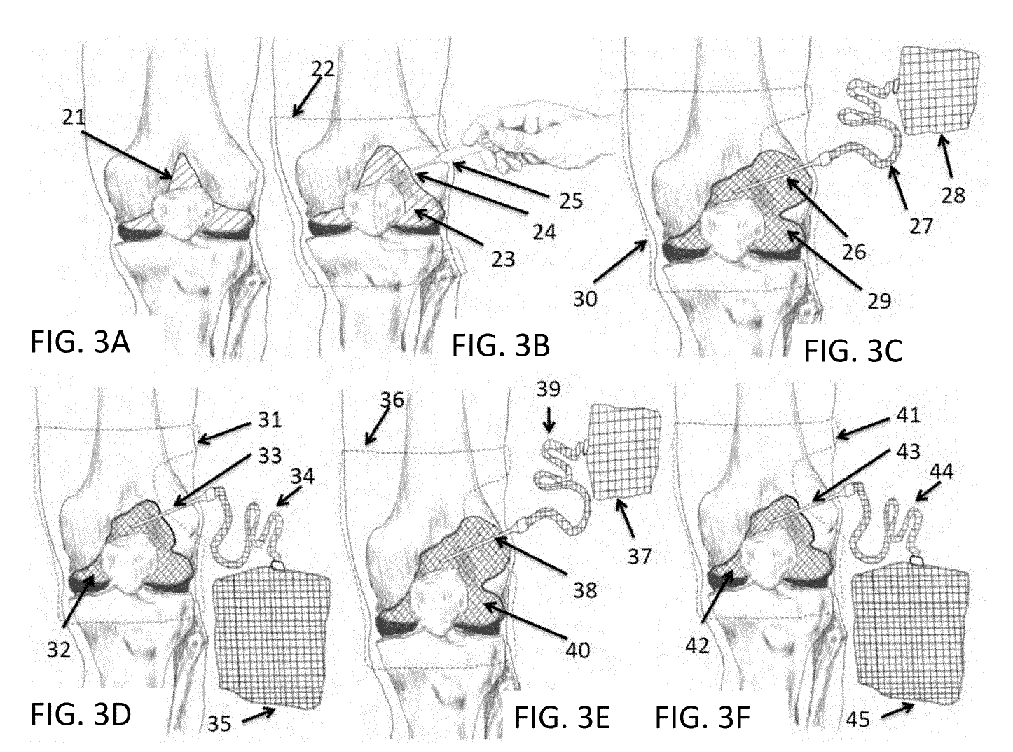

Two-way or tidal lavage decompresses the joint and simultaneously provides fluid for analysis, including biomarkers, as in one-way lavage as is shown in Examples 1 and 2, but the lavage fluid is extracted rather than allowed to leave the joint via endogenous pathways, making multiple lavage cycles possible. FIG. 3A, FIG. 3B, FIG. 3C, FIG. 3D, FIG. 3E, and FIG. 3F together illustrate the basic process of a cycle of pressure-assisted joint lavage. FIG. 3A shows a joint that could have minimal fluid or an effusion 21 (diagonal hatch). FIG. 3B shows how a compression brace is used to insert a lavage needle or catheter and shift fluid a predetermined portal where it becomes more accessible. A compression brace 21 (broken line) can be placed on the joint and the compression brace shifts fluid 23 (diagonal hatch) to the access window of the brace 22 (broken line). A lavage needle or catheter 24 on a syringe 25 is introduced through the access window and into the shifted synovial fluid 23 and the fluid is aspirated until fluid flow ceases. The syringe 25 is then removed and the needle or catheter 24 is left residing intraarticularly. FIG. 3C shows the infusion phase of tidal lavage. The intraarticular needle or catheter 26 is attached to a lavage fluid source either a syringe or in this case tubing 27 attached to a syringe, bottle or bag of lavage fluid 28. The compressive brace 30 can be released or relaxed to decrease intraarticular pressure so that joint can fill facilely with lavage fluid. The lavage fluid (crosshatch) in the bag 28 flows through the tubing 27 and needle 26 and into the synovial space of the joint where the lavage fluid 29 (crosshatch) fills the joint space including the peri-synovial soft tissues. The source of lavage fluid in this case a bag 28 can be, as examples, gravity fed, a pump or pressure system, a syringe, or a refilling syringe. During the infusion phase of joint lavage the tension from the 30 compression brace (broken line) can be released so that the joint can fill fully with lavage fluid at low pressure. In the case of a fabric or elastomeric brace, the straps or fasteners can be loosened or released; in the case of a pneumatic brace, the pressure can be decreased or released from the internal bladders.

FIG. 3D shows the aspiration phase of lavage. First the compression brace 31 is reengaged (from refastening straps in the mechanical brace or increasing pressure in the air bladders of the pneumatic brace) to compress the joint and thereby move the intraarticular lavage fluid 32 (crosshatch) to a site from which it can easily be drained. Under this pressure the lavage fluid 32 flows through the needle 33, the tubing 34, and into the collection receptacle, in this example case a bag 35 (crosshatch). This process can be assisted by gravity, a pump, a vacuum bottle, or a refilling syringe. The fluid in the receptacle or bag 35 can also be sent for previously mentioned analyses including biomarker profiles. This concludes one cycle of two-way or tidal lavage.

FIGS. 3E and 3F show how additional cycles of tidal lavage can be undertaken. In FIG. 3E, initiation of a second infusion cycle is illustrated, wherein the brace 36 is again relaxed or released, decreasing pressure within the joint. A new source of the lavage fluid in this case a bag 37 with fresh lavage fluid (crosshatch) is attached to the intraarticular needle or catheter 38, and the new lavage fluid (crosshatch) flows the bag 37 into the tubing 39, through the intraarticular needle 38, into the intraarticular space 40 that fills with lavage fluid (crosshatch).

FIG. 3F shows the aspiration cycle of tidal lavage. First the compression brace 41 is reengaged to compress the joint, and increase intraarticular pressure, and thereby move the intraarticular lavage fluid 42 (crosshatch) under the increased intraarticular pressure and direct it to the intra-articular compartment from which it will be drained. Under this pressure the intraarticular lavage fluid 42 flows through the needle 43, the tubing 44, and into a collection receptacle, in this case a bag 45 (crosshatch). This concludes the second cycle of two-way or tidal lavage.

As shown the lavage fluid source can be a bag or bottle or other lavage fluid source or can be provided using syringes. Although a reciprocating syringe can be advantageous for this procedure, a conventional syringe, an automatic syringe, or a refilling syringe (including a syringe with an aspiration spring on the plunger) can also be suitable. A reciprocating syringe, conventional syringe, refilling syringe, or automatic syringe can be used to first inject and then aspirate the lavage fluid in one cycle, or in multiple cycles with or without syringe exchanges. After this lavage, the fluid can be analyzed for biomarkers and other factors. After two-way or tidal lavage, the joint can be injected with corticosteroid, hyaluronan, or other therapeutic substance through the same needle that is then extracted.

Example 4—One and Two-Way Lavage Systems with Valves

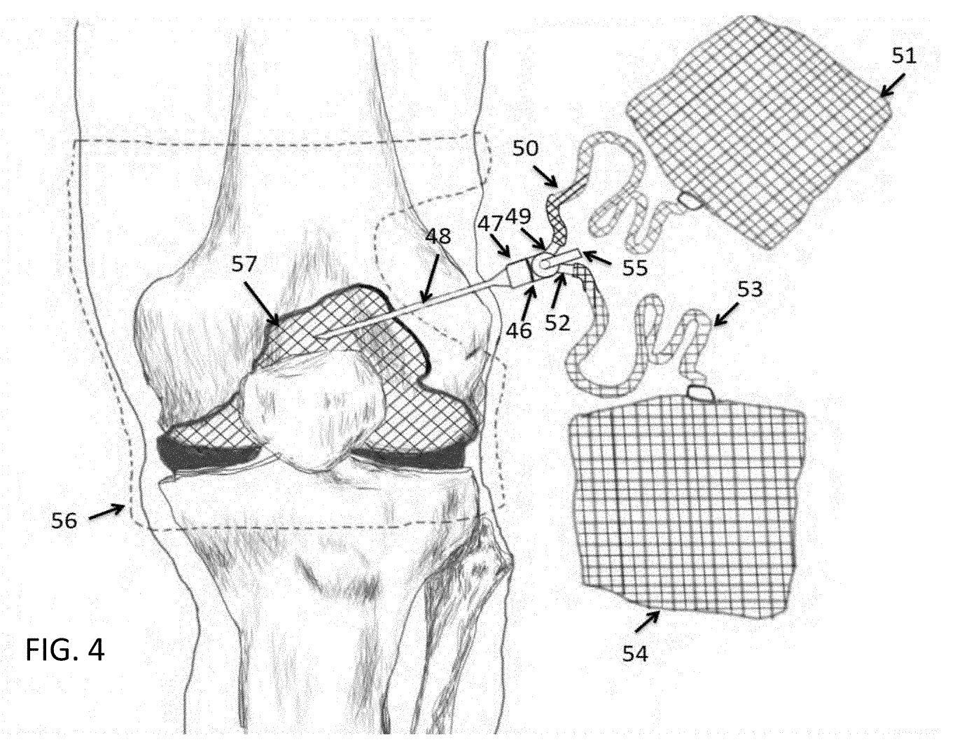

FIG. 4 demonstrates one embodiment of a lavage system with valves, in this case a 3-port stopcock 46 that permits lavage fluid to flow in and out of the knee in a sequential fashion. The stopcock 46 has a needle port 47 that accommodates a needle 48 or catheter that dwells intraarticularly. The needle 48 or catheter is inserted prior to addition of the stopcock in a manner completely conventionally as shown in FIGS. 1-3 or with the stopcock on and attached to a syringe. The stopcock has an input port 49 attached to a lavage source, in this case tubing 50 attached to a bag 51 or bottle, including a pressure bottle, of lavage fluid. The pressure to move the lavage fluid could be gravity, a pump, syringe a refilling syringe, or other means. The stopcock 46 has an output port 52 attached to a reservoir for spent lavage fluid, in this case tubing 53 leading to a bag 54 or bottle, which can include, for example, a vacuum bottle to create negative pressure. The stopcock 46 has a lever 55 or mechanism so that input port 49 or output port 52 can be alternatively continuous with the needle port 47 and catheter or needle 48. There is a compressive brace 56, e.g., either pneumatic as described by U.S. Pat. No. 7,468,048 to Meehan 2008 or mechanical as described by US Patent application to Band 2017. To cycle the lavage system as shown in FIG. 4, the compressive brace 56 is first relaxed or released so the intraarticular space 57 is low pressure and can expand. Higher pressure is created in the lavage reservoir, in this example a bag 51 with lavage fluid (crosshatch in bag) by compression, gravity, or a pump; the lavage fluid (crosshatch in tubing 50) flows into the input tubing 50. The stopcock lever 55 is moved so that the lavage fluid can flow through the input port 49 and stopcock 46, the needle port 47, the needle 48, and into the intraarticular space of the joint. The stopcock lever can be configured to enable the pressure and flow of the lavage fluid to be controlled. The intraarticular space 57 then fills with lavage fluid (crosshatch) to capacity, constituting half of the lavage cycle. At this point the stopcock lever 55 is moved so that the input port 49 is closed and the output port 52 is open. At this point the compressive brace 56 is reengaged increasing the pressure in the intraarticular space 57 and driving spent lavage fluid (crosshatch) towards the needle 48. The spent lavage fluid (crosshatch) moves into the needle 48, needle port 47 of the stopcock 46, through the output port 52, into the tubing 53. The spent lavage fluid (crosshatch) moves through the tubing 53 and into receptacle, in this example a bag 54, which then fills with spent lavage fluid. This constitutes one full cycle of the lavage process. A second cycle would be performed identically to above.

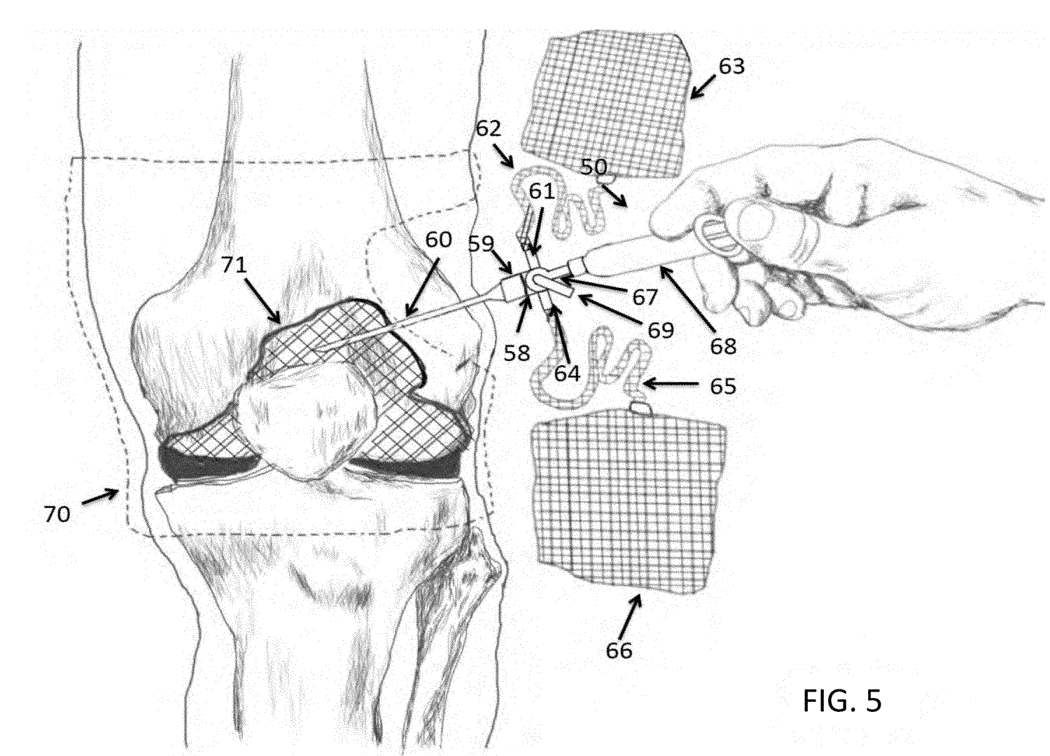

FIG. 5 shows a 4-port stopcock embodiment with a syringe. The stopcock 58 has a needle port 59 that accommodates a needle 60 or catheter that dwells intraarticularly. The needle 60 or catheter is inserted prior to addition of the stopcock 58 in a conventional manner as shown in FIGS. 1-3 or with the stopcock on and attached to a syringe. The stopcock has an input port 61 attached to a lavage source in this case tubing 62 attached to bag 63 or bottle, including a pressure bottle, of lavage fluid. The pressure to move the lavage fluid can be gravity, a pump, a refilling syringe, or other means. The stopcock 58 has an output port 64 attached to a reservoir for spent lavage fluid in this case tubing 65 leading to a bag 66 or bottle, including a vacuum bottle to create negative pressure. Finally, the stopcock 58 has a syringe port 67 that is attached via tubing or directly to a syringe 68. The stopcock 58 has a lever 69 or mechanism so that input port 61, output port 64, or needle port 59 can be alternatively continuous with the syringe port 67 and syringe 68. The stopcock lever can be configured to enable the flow and pressure of the moving fluid to be controlled. There is a compressive brace 70, e.g. either pneumatic as described by U.S. Pat. No. 7,468,048 to Meehan 2008 or mechanical as described by US Patent application to Band 2017. After using the compressive device to ensure accurate needle placement and complete pre-lavage evacuation of the joint, the lavage system is performed as shown in FIG. 5. The compressive brace 70 is first relaxed or released so the intraarticular space 71 is low pressure and can expand. The stopcock lever 69 is moved so that the syringe port 67 and input port 61 are continuous. The plunger of the syringe 68 is retracted and the syringe fills with lavage fluid. The lever 69 is then moved so that the needle port 59 and needle 60 are continuous with the syringe port 67 and syringe 68. The lavage fluid is then injected from the syringe 68, syringe port 67, stopcock 58, the needle port 59, the needle 60, and into the intraarticular space 71 of the joint. The intraarticular space 71 then fills with lavage fluid (crosshatch). This process of filling the syringe and injecting into the joint through the stopcock continues until the syringe is filled to capacity, constituting half of the lavage cycle. At this point the stopcock lever 69 is moved so that the input port 61 and output port 64 are closed and the needle port 59 is open and continuous with the syringe port 67 and syringe 68. At this point the compressive brace 70 is reengaged increasing the pressure in the intraarticular space 71 and driving spent lavage fluid (crosshatch) towards the needle 60. The spent lavage fluid (crosshatch) moves into the needle 60, needle port 59 of the stopcock 58, into the syringe port 67 and syringe 68 that then fill with spent lavage fluid. The stopcock lever 69 is then moved so that the syringe port 67 and syringe 68 are continuous with the output port 64. The spent lavage fluid is then injected out of the syringe 68, into the syringe port 67, stopcock 58, and output port 64, into the tubing 65. The spent lavage fluid (crosshatch) moves through the tubing 65 and into the receptacle, in this case a bag 66, which then fills with spent lavage fluid (crosshatch). This process is then repeated until the joint is completely decompressed. This constitutes one full cycle of the lavage process using the 4-port stopcock. Second and subsequent cycles can be performed identically to above. Although a reciprocating syringe can be advantageous for this procedure, a conventional syringe, automatic syringe, or a refilling syringe (including a syringe with a aspiration spring on the plunger) can also function for this purpose.

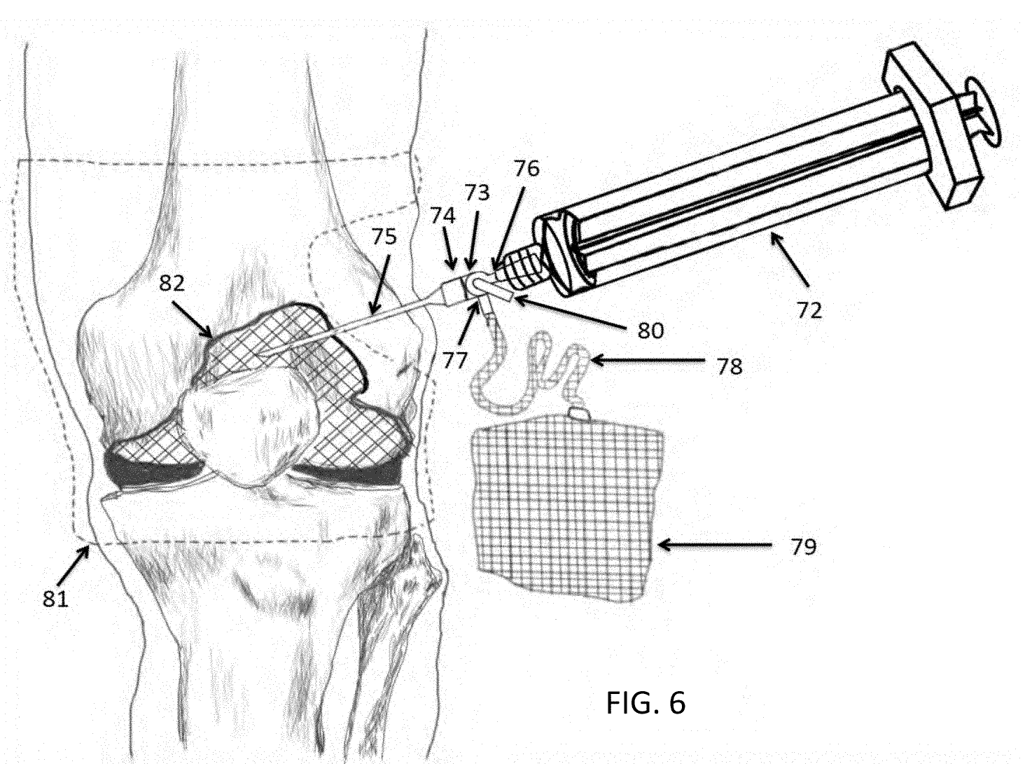

FIG. 6 shows a 3-port stopcock embodiment with a syringe 72 that permits lavage fluid to flow in and out of the knee in a sequential fashion. The stopcock 73 has a needle port 74 that accommodates a needle 75 or catheter that dwells intraarticularly. The needle 75 or catheter is inserted prior to addition of the stopcock in a manner completely conventionally as shown in FIGS. 1-3 or with the stopcock on and attached to a syringe. The stopcock has an input port 76 attached to a lavage source in this case a large syringe 72, for example 10 ml to 150 ml. The pressure to move the lavage fluid is generated by the syringe. The stopcock 74 has an egress port 77 attached to a reservoir for spent lavage fluid in this case tubing 78 leading to a bag 79 or bottle, including a vacuum bottle to create negative pressure. The stopcock 73 has a lever 80 or mechanism so that the input port 76 can be alternatively continuous with the needle port 74 and catheter or needle 75 or with the egress port 77 for spent lavage fluid. The stopcock lever can additionally be designed to enable the fluid pressure to be controlled. There is a compressive brace 81, e.g., either pneumatic as described by U.S. Pat. No. 7,468,048 to Meehan 2008 or mechanical as described by US Patent application to Band 2017. To cycle the lavage system as shown in FIG. 6, the compressive brace 81 is first relaxed or released so that the intraarticular space 82 is low pressure and can expand. Higher pressure is created in the lavage source in this case a syringe 72, and the lavage fluid flows into the input port 76. The stopcock lever 80 is moved so that the lavage fluid can flow through the input port 76 and stopcock 73, the needle port 74, the needle 75, and into the intraarticular space of the joint 82. The intraarticular space 82 then fills with lavage fluid (crosshatch) to capacity, constituting half of the lavage cycle. At this point the stopcock lever 80 is left open so that the input port 76 is continuous with the needle port 74 and needle 75. At this point the compressive brace 81 is reengaged increasing the pressure in the intraarticular space 82 and driving spent lavage fluid (crosshatch) towards the needle 75. The spent lavage fluid (crosshatch) moves into the needle 75, needle port 74 of the stopcock 73, through the input port 76, into the syringe 72. The stopcock lever 80 is then moved so that the input port 76 is continuous with the output port 77. The plunger of the lavage syringe 72 is compressed and the spent lavage fluid (crosshatch) moves through the tubing 78 and into receptacle, in this example a bag 79, which then fills with spent lavage fluid. This constitutes one full cycle of the lavage process. Second and subsequent cycles, if desired, can be performed identically to above by providing a new syringe 72 filled with fresh lavage fluid. This process can also be completed with a syringe without a stopcock.

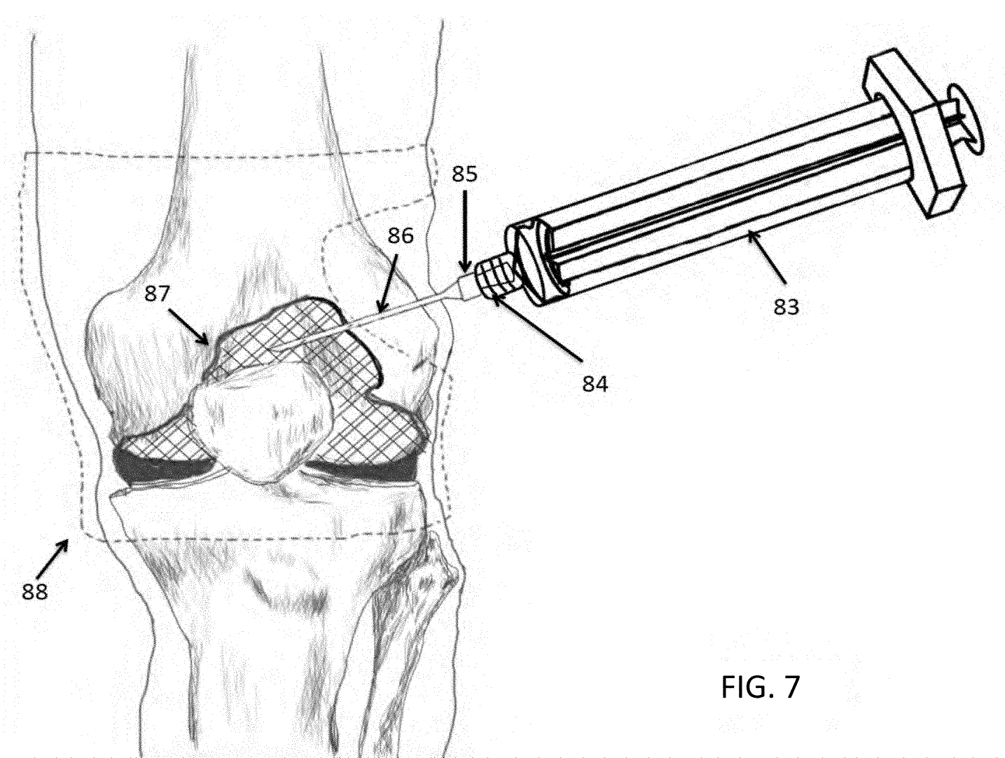

FIG. 7 shows that simple lavage can also performed with a syringe or a series of syringes without a stopcock or valve. The needle or catheter is introduced as detailed in FIGS. 1-3. A syringe 83 has a needle fitting 84 that is attached to the syringe fitting 85 of the needle 86 that has been placed into the intraarticular space 87. There is a compressive brace 88, e.g., either pneumatic as described by U.S. Pat. No. 7,468,048 to Meehan 2008 or mechanical as described by US Patent application to Band 2017. The pressure to move the lavage fluid is generated by the syringe. To cycle the lavage system as shown in FIG. 7, the compressive brace 88 is first relaxed or released so that the intraarticular space 87 is low pressure and can expand. Higher pressure is created in the lavage source in this case a syringe 83; the lavage fluid flows into the needle 86, into the intraarticular space of the joint 87, and into the peri-articular soft tissues. The intraarticular space 87 then fills with lavage fluid (crosshatch) to capacity, constituting half of the lavage cycle. At this point the compressive brace 88 is reengaged increasing the pressure in the intraarticular space 87 and driving spent lavage fluid (crosshatch) towards the needle 86. The spent lavage fluid (crosshatch) moves into the needle 86 and into the syringe 83. This constitutes one full cycle of the lavage process. Second and subsequent cycles, if desired, can be performed identically to above by providing a new syringe 83 filled with fresh lavage fluid.

Example 5—One and Two-Way Lavage Systems with Check Valves

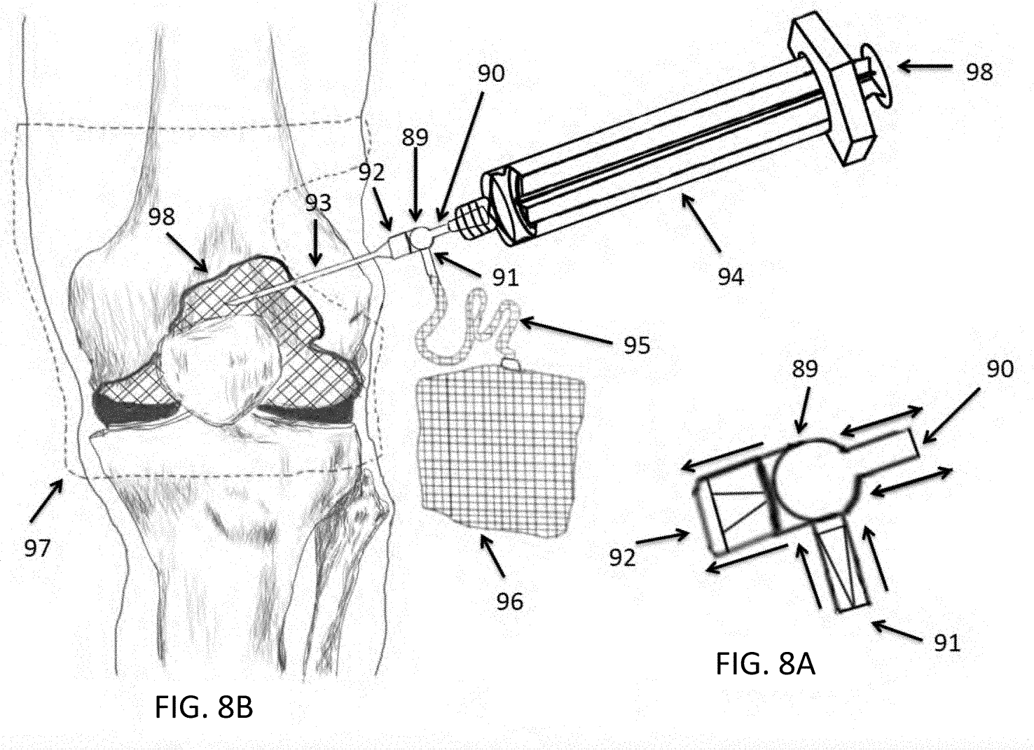

FIG. 8A and FIG. 8B together depict an embodiment that facilitates high volume one-way flow into the intraarticular space using a series of check valves. First, a needle or catheter is introduced intraarticularly as described in connection with FIGS. 1-3. FIG. 8A show a close-up of the 3 port double check valve apparatus 89 with a syringe port 90 without a check valve that permits two-way flow (double arrow), a lavage fluid port 91 with a one-way check valve that permit one-way flow (arrows) only into the fitting, and a needle port 92 with a check valve that only permits flow out of the port (arrows). FIG. 8B shows how the system functions. First, the plunger 98 of the syringe 94 is pulled backward creating a vacuum. The needle port 92 is blocked by the one-way check valve and there is no flow. However, the lavage fluid port 91 does permit lavage fluid (crosshatch) to flow from the lavage source, in this example a bag 96, wherefrom the lavage fluid flows into the tubing 95, lavage port 91, into the apparatus 89, the syringe fitting 90 and into the syringe 94. Before injecting fluid the compressive brace 97 is relaxed or released so that the intraarticular space 98 can fill with lavage fluid. After the syringe 94 is filled with lavage fluid, the syringe plunger 98 is depressed, the lavage fluid flows from the syringe 94, into the syringe fitting 90, apparatus 89, needle fitting 92 where now the check valve permits one-way flow into the needle 93, and from the needle 93 into the intraarticular space 98 which fills with lavage fluid (crosshatch). This process can be repeated until the joint is sufficiently distended with lavage fluid to achieve therapeutic goals, such as hydro-dissection and the gentle disruption of capsular adhesions. The lavage fluid can be left in the joint for one-way lavage, or can then be aspirated for two-way lavage.

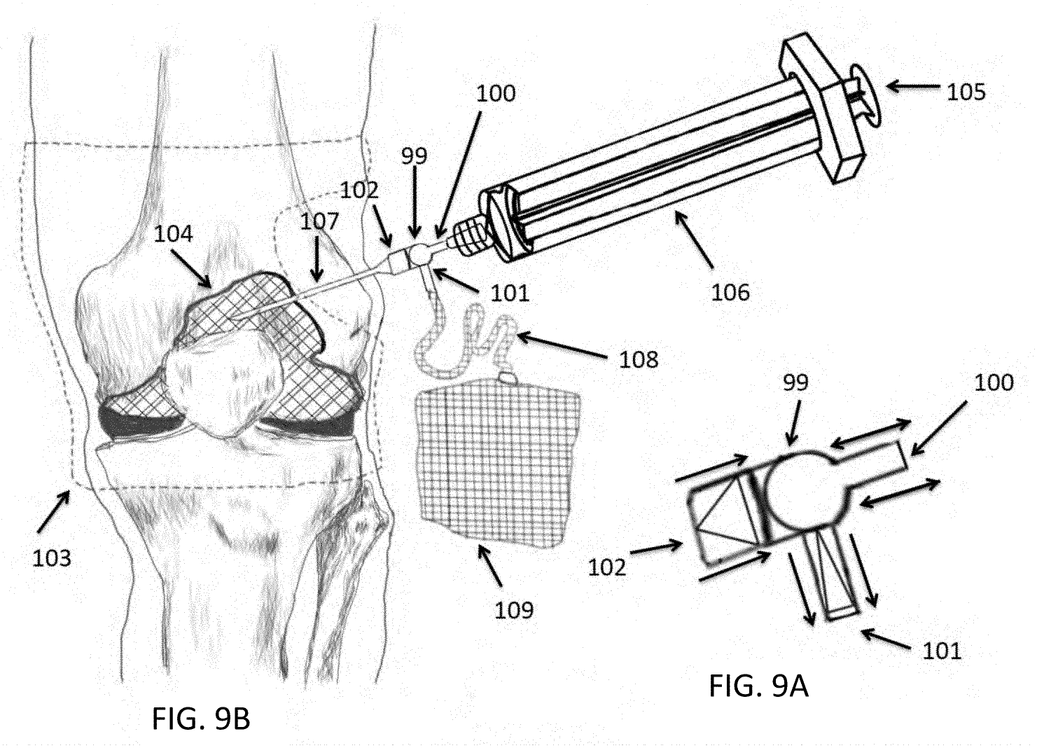

FIG. 9A and FIG. 9B together depict an example embodiment that facilitates high volume one-way flow out of the intraarticular space using a series of check valves. This embodiment can be placed when the joint becomes completely distended with lavage fluid as in FIG. 8 or can be used for large volume arthrocentesis. First, a needle or catheter is introduced intraarticularly as described in connection with FIGS. 1-3. The compression brace 103 is then reengaged to increase pressure in the lavage fluid (crosshatch) in the intraarticular space 104 and surrounding soft tissues. FIG. 9A show a close-up of the 3 port double check valve apparatus 99 with a syringe port 100 without a check valve that permits two-way flow (double arrow), a spent lavage fluid port 101 with a one-way check valve that permit one-way flow (arrows) only out of the port 101, and a needle port 102 with a check valve that only permits flow into the port 102 (arrows). FIG. 9B shows how the system functions. First, the plunger 105 of the syringe 106 is pulled backward creating a vacuum. The spent lavage port 101 is blocked by the one-way check valve and there is no flow. However, the needle port 102 does permit intraarticular lavage fluid (crosshatch) in the intraarticular space 104 to flow into the needle 107 and then the needle port 102 and apparatus, into the syringe fitting 100 and fills the syringe 106. After the syringe 106 is filled with spent lavage fluid, the syringe plunger 105 is depressed, the lavage fluid flows from the syringe 105, into the syringe fitting 100, apparatus 99, spent lavage fluid fitting 101 where now the check valve permits one-way flow into the spent lavage tubing 108, and spent lavage receptacle, in this case a bag 109. This is repeated until the joint is completely decompressed at which time the compressive brace 103 is released.

Example 6—Tubing and Skin Anchored Lavage Systems

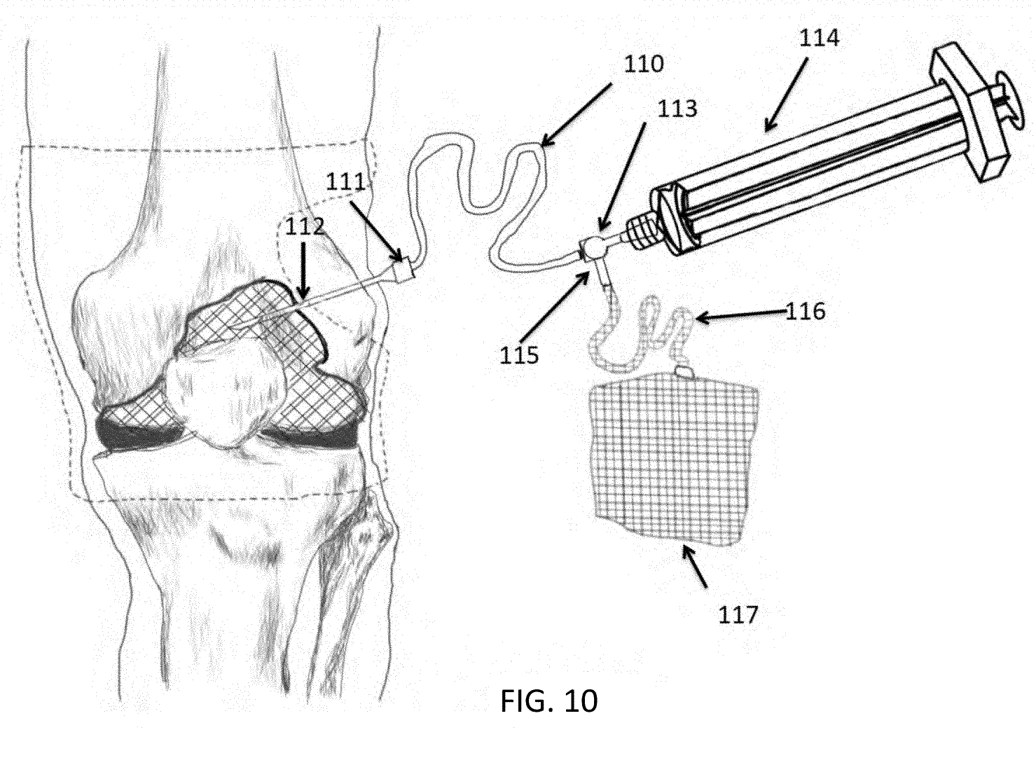

A potential problem with these manipulations in some applications is that the syringe apparatus (syringe, syringe and stopcock, or syringe and check valve system) must be hand-held and every movement of the syringe moves the needle in the joint, and that can cause trauma to cartilage and synovium. To reduce this movement associated with syringe manipulation, a length of extension tubing can be placed between the intraarticular needle and the lavage syringe system. FIG. 10 illustrates an example embodiment with tubing so that the needle or catheter does not move with manipulations of the syringe and cycling of the lavage system. The tubing 110 is attached to the needle fitting 111 so that needle 112 does not move significantly during manipulations of other components of the system. The distal end of the tubing 110 is attached either to a valve system (stopcock or check valve system) 113 or directly to a syringe 114. A fitting 115 on the valve system 113 can also be connected by tubing 116 to a reservoir(s) 117 for lavage fluid and/or spent lavage fluid.

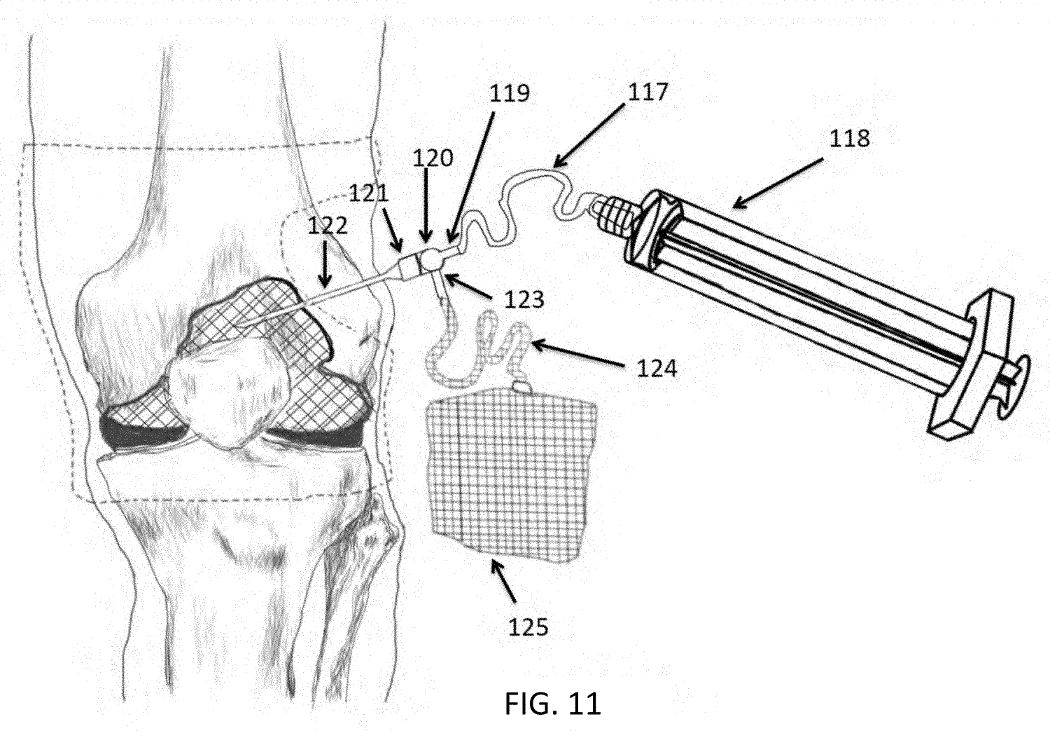

FIG. 11 is another example embodiment with tubing. The tubing 117 is attached to the syringe 118 and fitting 119 on the valve system 120 with a needle fitting 121 and catheter or needle 122 so that needle 22 does not move significantly with manipulations of the syringe. A fitting 123 on the valve system (stopcock or check valve system) 120 can also be connected by tubing 124 to a reservoir(s) 125 for lavage fluid and/or spent lavage fluid.

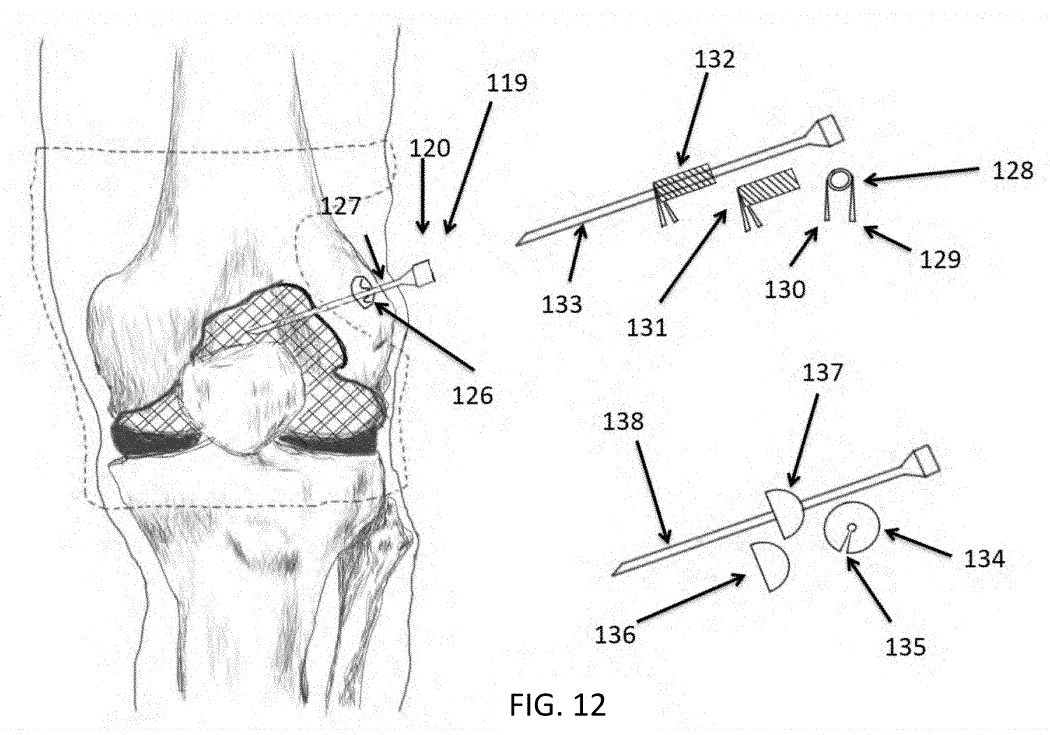

The intraarticular needle can be held with the hand in position or can be secured with tape or medical adhesive. FIG. 12 illustrates example configurations to fix the lavage needle to the skin. A skin fixation device 126 fixes the catheter or needle 127 to the skin so that the needle tip remains at the same depth during the manipulations of lavage. In one example embodiment an adjustable collar (depth marker) can be used, the position adjusted on the length of the needle and then secured to the skin mechanically, including surgical tape or adhesive. The adjustable collar 128 can be a metal spring-based device with arms 129 and 130 that can be pinched to loosen the springe device by dilating its circumference. The arms can be at one end 131 and can be used to tape the device to the skin. The adjustable collar (depth marker) 132 when the arms are pinched together can move along the shaft of the needle 133 and released to bind to the needle at the skin surface. It can also comprise a releasable plastic clamp.

Alternatively, the skin fixation collar can be a broadly hemispherical device 134 of plastic or foam or similar material, with an access cutaway 135 so it can be placed around the catheter or needle. An adhesive surface 136 can bind the collar 137 to the skin at a defined position on the needle shaft 138.

Example 7. Two-Catheter Systems.

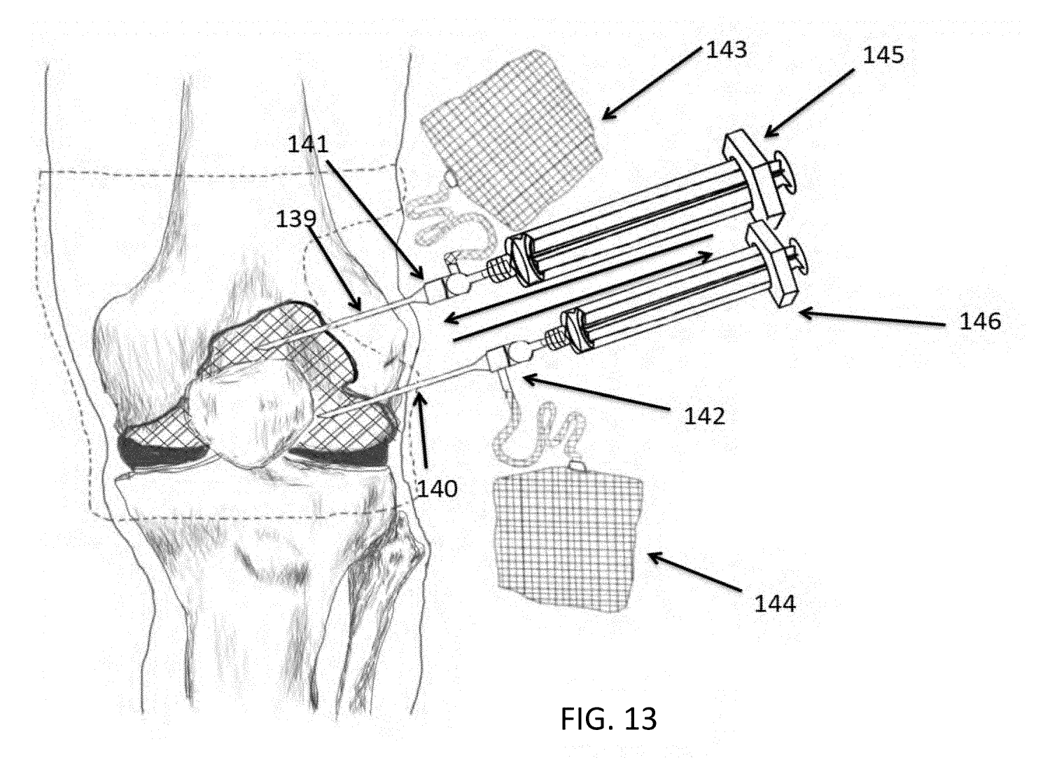

Although all the systems described above assume a one-needle percutaneous system where one needle is used for both lavage input into and lavage fluid output from the joint, a two-needle or two-catheter system can also be used. FIG. 13 illustrates a two-catheter lavage system where one intraarticular needle 139 or catheter is for a fresh lavage fluid input port, and a second intraarticular needle 140 or catheter is a spent lavage output port. In this system there can be opposing valves (check valves or stop cocks) 141 and 142, fresh and spent lavage fluid reservoirs 143 and 144, and separate actuating syringes 145 and 146. During input into the joint, the lavage output needle or tubing can be clamped, or closed with a stopcock or other active or passive closure system, or simply left inactive, and then after full joint distention opened to drain the joint of lavage fluid as completely described in the earlier embodiments.

Example 8—Alternative Catheter Systems

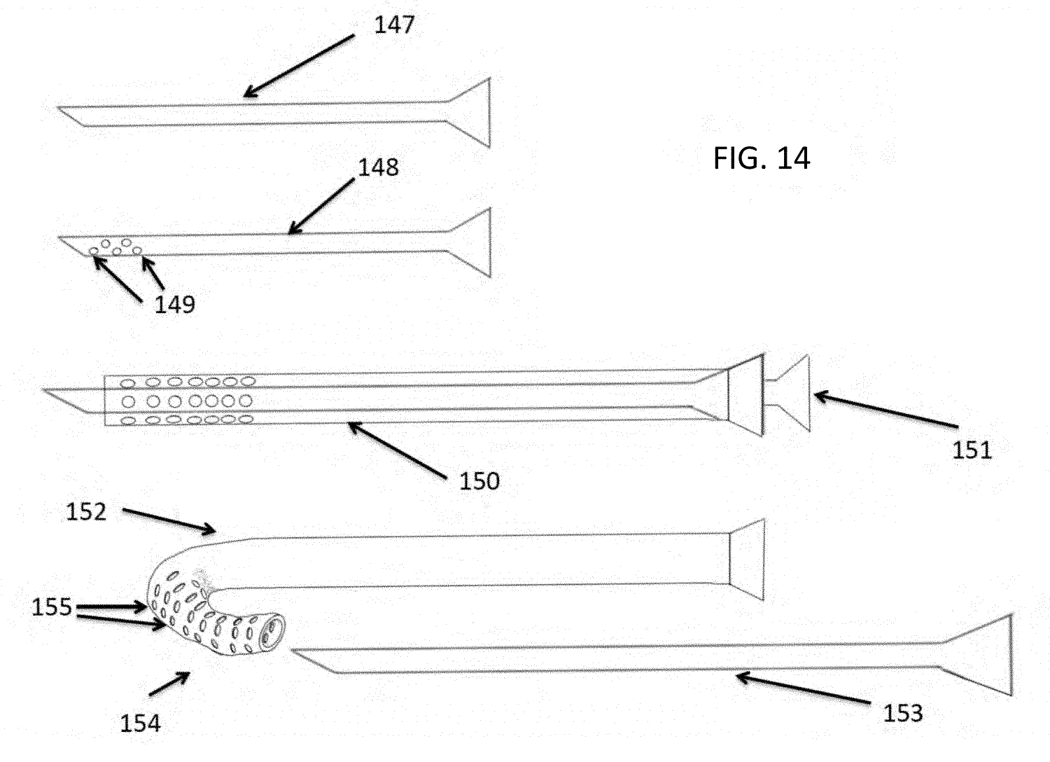

Although the intraarticular lavage systems above were described as using a conventional needle, other needle and catheter types can have beneficial effects on joint lavage. FIG. 14 shows several example embodiments for needle and catheters for lavage systems. Any of these needles and catheters can be directly introduced into the joint as with a conventional needle or catheter, but since many have larger diameters, it can be first advantageous to anesthetize the joint and partially dilate the joint with a conventional needle 147, then place the specialty needle or catheter into the partially dilated joint. One type of needle that can be beneficial is a specialty needle 148 with side windows or fenestrations 149 that permit multiple exit and ingress openings for movement of synovial fluid, which can be beneficial as the synovial membrane can block a single port. A plastic catheter 150 can also be introduced into the joint either on the outside of an introducer needle 151 or through the lumen of an introducer needle. For many applications a pig-tailed or curved catheter 152 can be useful that, after removal of the introducer needle 153 curls up 154 inside the joint and thus can be more stable during the manipulations for lavage. Multiple side ports or fenestrations 155 along the length of the needle or catheter can facilitate drainage by circumventing blockage of flow by overgrown synovium, and can be advantageous in this system. Alternatively, the Selinger technique can be used, where a wire is introduced into the joint through an introducer needle, a dilator is placed over the wire, and finally, after the dilator is removed, a catheter is placed over the wire into the joint, and the wire is then removed leaving intraarticular catheter for joint lavage.

Example 9—Lavage followed by Injection of a Therapeutic Substance

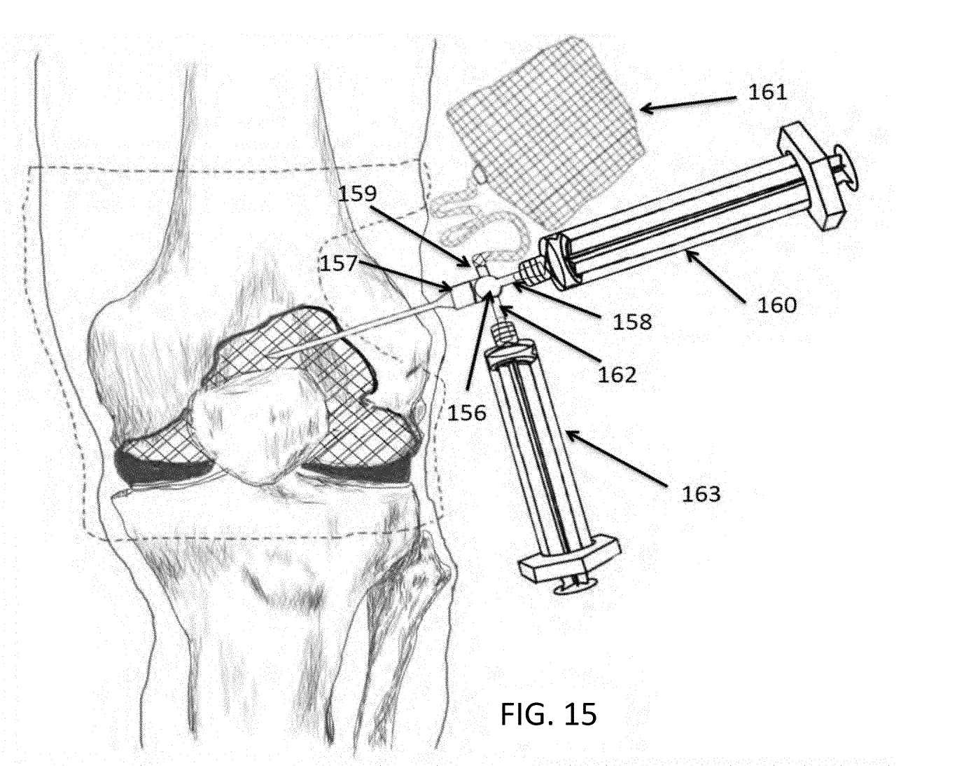

FIG. 15 shows the elements of using a multiport valve for lavage followed by injection of a therapeutic substance. A three or four port valve system 156 with (1) a 1st port for attaching the needle 157, (2) a 2nd port 158 and, optionally, 3rd port 159 with syringe 160 and lavage source 161 permitting lavage cycles as previous described, and (3) a 3rd or 4th port 162 for injecting a therapeutic substance (syringe) 163 would have advantages for this system. Although a stopcock would function as previously discussed, a valve system that permits free exchange between the 1st port and 2nd port with a closed 3rd port during the lavage, and free exchange between the 1st port and 3rd port with a closed 2nd port during the injection of the therapeutic substance can also be advantageous. A special connector without needles can be configured such that when the lavage syringe is attached the 2nd port is opened and the 3rd port is closed, and when the lavage syringe is removed the 2nd port closes and the 3rd port automatically opens. Alternatively, when fluid is injected through the syringe on the 3rd port, the 2nd port mechanically closes either driven directly by the plunger of the syringe or by pressure from flow through the syringe used to inject the therapeutic substance.

Example 10—One-Way or Two-Way Intrasynovial or Subsynovial Membrane Lavage

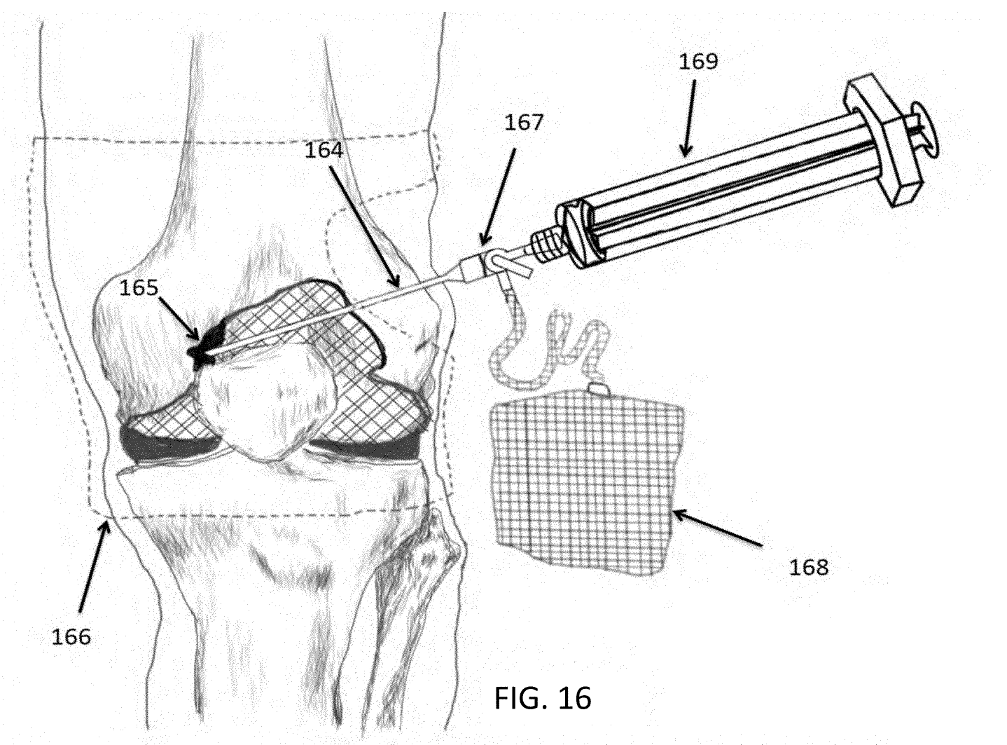

FIG. 16 demonstrates the basic elements for subsynovial membrane lavage. In some circumstances it can be useful to lavage or inject the synovial membrane instead of the intraarticular space. In this case, ultrasound, fluoroscopy, or MRI can be used to direct the needle 164 into the synovial membrane 165 and the synovial membrane 165 itself can be injected, dissected, lavaged, or a combination thereof, using any of the above systems. A large intrasynovial or subsynovial pocket 165 can be created to prolong the effects of intraarticular therapies, providing for true slow release of the substance. Alternatively, a sclerosing or antiproliferative substance can be injected intrasynovaial and subsynovially to reduce hypertrophic tissue. Other elements including compressive brace 166, valve system 167, lavage fluid source 168, and lavage syringe 169 are as previously described above and can be included in this example embodiment.

Example 10—Fluids for Lavage

Although normal saline is ordinarily be used for intraarticular lavage, other fluids can be used included lactated Ringer's solution, various solutions of glucose or saline or both, hypotonic or hypertonic solutions, solutions with local anesthetics, antibiotic containing solutions, glucosamine containing solutions, autologous serum or plasma and its components, growth factors, antibiotics, acid or basic or pH adjusted solutions, chelating agents such as EDTA and related compounds to remove calcium deposits, biologic response modifiers, stem cells and solutions of hyaluronic acid and its derivatives. Lavage fluids can be discarded or saved and analyzed for bacteria, fungi, virus, crystals, white blood cells, cytokines, biomarkers, cartilage breakdown products, or other molecules of interest. Analytes found in the lavage fluid can be compared to analytes found in the neat synovial fluid extracted during the initial compression procedure for accurate needle placement. After lavage, but before the needle or catheter is removed, the joint can be injected with corticosteroids, local anesthetics, antibiotics, glucosamine containing solutions, autologous serum or plasma and its components, stem cells, biologic response modifiers, small molecule drugs, or hyaluronic acid solutions and derivatives.

The following references can facilitate understanding of the present invention, and are incorporated herein by reference:

Zhang W, Moskowitz R W, Nuki G, Abramson S, Altman R D, Arden N, Bierma-Zeinstra S, Brandt K D, Croft P, Doherty M, Dougados M, Hochberg M, Hunter D J, Kwoh K, Lohmander L S, Tugwell P., McAlindon T E, Bannuru R R, Sullivan M C, Arden N K, Berenbaum F, Bierma-Zeinstra S M, Hawker G A, Henrotin Y, Hunter D J, Kawaguchi H, Kwoh K, Lohmander S, Rannou F, Roos E M, Underwood M. OARSI guidelines for the non-surgical management of knee osteoarthritis. Osteoarthritis and Cartilage 22 (2014) 363e388.

American Academy of Orthopaedic Surgeons. TREATMENT OF OSTEOARTHRITIS OF THE KNEE. EVIDENCE-BASED GUIDELINE 2nd EDITION. https://www.aaos.orecc_files/aaosoreresearch/guidelines/treatmentofosteoarthritisofthekneeguideline.pdf accessed Jan. 20, 2018

NICE. National Institute for Health and Care Excellence. Osteoarthritis: care and management. Clinical guideline [CG177]. February 2014. https://www.nice.org.uk/guidance/cg177 accessed Jan. 20, 2018

Bhaysar T B, Sibbitt W L. Jr, Band P A, Cabacungan R J, Moore T S, Salayandia L C, Fields R A, Kettwich S K., Roldan L P, Emil N S, Fangtham M, Bankhurst A D. Improvement in diagnostic and therapeutic arthrocentesis via constant compression. Clin Rheumatol. 2017 Sep. 14. doi: 10.1007/510067-017-3836-x. [Epub ahead of print] PMID: 28913649

Meehan R, Wilson C, Hoffman E, Regan E, Altimier L. Ultrasound quantification of fluid shifts in the knees of arthritis patients before and after inflation of a pneumatic compressive device. Arthritis Rheum 2015; 67 (Abstract Supplement):180.

Yaqub S, Sibbitt W L Jr, Band P A, Bennett J F, Emil N S, Fangtham M, Fields R A, Hayward W A, Kettwich S K, Roldan L P, Bankhurst A D. Can Diagnostic and Therapeutic Arthrocentesis be Successfully Performed in the Flexed Knee?—Journal of Clinical Rheumatology—J Clin Rheumatol. 2018 Feb. 7. doi: 10.1097/RHU.0000000000000707. [Epub ahead of print] PMID: 29424762

Siparsky P, Ryzewicz M, Peterson B, Bartz R. Arthroscopic treatment of osteoarthritis of the knee: are there any evidence-based indications? Clin Orthop Relat Res. 2007 February; 455:107-12. Review.

Lundsgaard C, Dufour N, Fallentin E, Winkel P, Gluud C.Intra-articular sodium hyaluronate 2 mL versus physiological saline 20 mL versus physiological saline 2 mL for painful knee osteoarthritis: a randomized clinical trial. Scand J Rheumatol. 2008 March-April; 37(2):142-50.

Dawes P T, Kirlew C, Haslock I. Saline washout for knee osteoarthritis: results of a controlled study. Clin Rheumatol. 1987; 6:61-3.

Bradley J D, Heilman D K, Katz B P, Gsell P, Wallick J E, Brandt K D. Tidal irrigation as treatment for knee osteoarthritis: a sham-controlled, randomized, double-blinded evaluation. Arthritis Rheum. 2002 January; 46(1):100-8.

Chang R W, Falconer J, Stulberg S D, Arnold W J, Manheim L M, Dyer A R A randomized, controlled trial of arthroscopic surgery versus closed-needle joint lavage for patients with osteoarthritis of the knee. Arthritis Rheum. 1993 March; 36(3):289-96.

Ayral X. Arthroscopy and joint lavage. Best Pract Res Clin Rheumatol. 2005; 19:401-15.

Kalunian K C, Moreland L W, Klashman D J, Brion P H, Concoff A L, Myers S, Singh R, Ike R W, Seeger L L, Rich E, Skovron M L Visually-guided irrigation in patients with early knee osteoarthritis: a multicenter randomized, controlled trial. Osteoarthritis Cartilage. 2000; 8:412-8.

Ike R W. Tidal irrigation in septic arthritis of the knee: a potential alternative to surgical drainage. J Rheumatol. 1993; 20:2104-11.

Ike R W, Arnold W J, Rothschild E W, Shaw H L.Tidal irrigation versus conservative medical management in patients with osteoarthritis of the knee: a prospective randomized study. Tidal Irrigation Cooperating Group. J Rheumatol. 1992 May; 19(5):772-9.

Caporali R, Rossi S, Montecucco C. Tidal irrigation in Milwaukee shoulder syndrome. J Rheumatol. 1994; 21:1781-2.

Hilliquin P, Le Devic P, Menkes C J. Comparison of the efficacy of nonsurgical synovectomy (synoviorthesis) and joint lavage in knee osteoarthritis with effusions. Rev Rhum Engl Ed. 1996 February; 63(2):93-102.

Meijer M C, van Weeren P R, Rijkenhuizen A B.

Clinical experiences of treating septic arthritis in the equine by repeated joint lavage: a series of 39 cases. J Vet Med A Physiol Pathol Clin Med. 2000; 47:351-65.

Ravaud P, Moulinier L, Giraudeau B, Ayral X, Guerin C, Noel E, Thomas P, Fautrel B, Mazieres B, Dougados M. Effects of joint lavage and steroid injection in patients with osteoarthritis of the knee: results of a multicenter, randomized, controlled trial. Arthritis Rheum. 1999 March; 42(3):475-82.

Hilliquin P, Le Devic P, Menkes C J. Comparison of the efficacy of nonsurgical synovectomy (synoviorthesis) and joint lavage in knee osteoarthritis with effusions. Rev Rhum Engl Ed. 1996 February; 63(2):93-102.

Edelson R, Burks R T, Bloebaum R D. Short-term effects of knee washout for osteoarthritis. Am J Sports Med. 1995 May-June; 23(3):345-9.

Sornay-Soares C, Job-Deslandre C, Kahan A. Joint lavage for treating recurrent knee involvement in patients with juvenile idiopathic arthritis. Joint Bone Spine. 2004 July; 71(4):296-9.

Frias G, Caracuel M A, Escudero A, Rumbao J, Perez-Gujo V, del Carmen Castro M, Font P, Gonzalez J, Collantes E. Assessment of the efficacy of joint lavage versus joint lavage plus corticoids in patients with osteoarthritis of the knee. Curr Med Res Opin. 2004 June; 20(6):861-7.

Smith M D, Wetherall M, Darby T, Esterman A, Slavotinek J, Roberts-Thomson P, Coleman M, Ahern M J. A randomized placebo-controlled trial of arthroscopic lavage versus lavage plus intra-articular corticosteroids in the management of symptomatic osteoarthritis of the knee. Rheumatology (Oxford). 2003 December; 42(12):1477-85. Epub 2003 Jul. 16.

Vad V B, Bhat A L, Sculco T P, Wickiewicz T L. Management of knee osteoarthritis: knee lavage combined with hylan versus hylan alone. Arch Phys Med Rehabil. 2003 May; 84(5):634-7.

McCain J P, Balazs E A, de la Rua H. Preliminary studies on the use of a viscoelastic solution in arthroscopic surgery of the temporomandibular joint. J Oral Maxillofac Surg. 1989 November; 47(11):1161-8.

Tulamo R M. Comparison of high-performance liquid chromatography with a radiometric assay for determination of the effect of intra-articular administration of corticosteroid and saline solution on synovial fluid hyaluronate concentration in horses. Am J Vet Res. 1991 December; 52(12):1940-4.

Saari H, Tulamo R M, Konttinen Y T, Sorsa T. Methylprednisolone acetate induced release of cartilage proteoglycans: determination by high performance liquid chromatography. Ann Rheum Dis. 1992 February; 51(2):214-9.

Brusie R W, Sullins K E, White N A 2nd, Coffin P C, Parker G A, Anver M R, Rosenberger J L. Evaluation of sodium hyaluronate therapy in induced septic arthritis in the horse. Equine Vet J Suppl. 1992 February; (11):18-23.

Sato S, Ohta M, Ohki H, Kawamura H, Motegi K. Effect of lavage with injection of sodium hyaluronate for patients with nonreducing disk displacement of the temporomandibular joint. Oral Surg Oral Med Oral Pathol Oral Radiol Endod. 1997 September; 84(3):241-4.

Raza K, Lee C Y, Pilling D, Heaton S, Situnayake R D, Carruthers D M, Buckley C D, Gordon C, Salmon M. Ultrasound guidance allows accurate needle placement and aspiration from small joints in patients with early inflammatory arthritis. Rheumatology (Oxford). 2003 August; 42(8):976-9. Epub 2003 Apr. 16.

Kraus V B, Huebner J L, Fink C, King J B, Brown S, Vail T P, Guilak F.

Urea as a passive transport marker for arthritis biomarker studies. Arthritis Rheum. 2002 February; 46(2):420-7.

Ratcliffe A, Shurety W, Caterson B. The quantitation of a native chondroitin sulfate epitope in synovial fluid lavages and articular cartilage from canine experimental osteoarthritis and disuse atrophy. Arthritis Rheum. 1993 April; 36(4):543-51.

Petersson I F, Sandqvist L, Svensson B, Saxne T. Cartilage markers in synovial fluid in symptomatic knee osteoarthritis. Ann Rheum Dis. 1997 January; 56(1):64-7.

U.S. Pat. No. 3,957,052 to Topham 1976 5. Pumping Syringe. 128/278.

U.S. Pat. No. 4,098,276 to Bloom 1978 7. Syringe Pumping Handle Grip and Method of Assembling Same. 128/215.

U.S. Pat. No. 4,314,586 to Folkman 1980 2. Disposable Valve. 137/625.47.

U.S. Pat. No. 4,595,102 to Cianci 1986 6. Kit for Performing a Medical Procedure. 206/572.

U.S. Pat. No. 4,872,866 to Davis 1989 10. Medical Lavage Apparatus. 604/227.

U.S. Pat. No. 5,306,237 to Clement 1994 4. Disposable Lavage. 604/30.

U.S. Pat. No. 5,330,424 to Palmer 1994 7. Medical Lavage Apparatus and Methods. 604/28.

U.S. Pat. No. 5,836,907 to Campbell 1998 11. Disposable Gastric Lavage Kit. 604/27.

U.S. Pat. No. 5,972,909 to Di Napoli. 1999 10. Hyaluronic Acid and Corresponding Salts for the Preparation of an Aqueous Solution Useful as Intra-Articular Lavage Liquid.

U.S. Pat. No. 7,468,048 to Meehan 2008.

U.S. Pat. No. 5,964,728 to Lln. 1999 10. Synchronous Vitreous Lavage Device for Opthalmology and An Ophthalmologic Lavage System Using the Same. 604/30

U.S. Pat. No. 6,245,046 to Sibbitt 2001 6. Reciprocating Syringes. 604/191.

U.S. Pat. No. 6,371,934 B1 to Jackson. 2002 4. Irrigation System and Tip with Debrider. 604/35.

Band, P A, Wisniewski, H G, Kraus, V B (2014): U.S. Pat. No. 8,883,427 Quantifying local inflammatory activity and its use to predict disease progression and tailor treatments.

U.S. Pat. No. 6,527,760 to Vad. 2003 4. Out-Patient Joint Lavage Kit and Protocol. 604/512.

US Utility Application Number 12899543-2010 to Sibbitt 2011.

The present invention has been described in connection with various example embodiments. It will be understood that the above description is merely illustrative of the applications of the principles of the present invention, the scope of which is to be determined by the claims viewed in light of the specification. Other variants and modifications of the invention will be apparent to those skilled in the art.

What is claimed is:

1. A joint lavage kit comprising:
(a) an external compression device;
(b) a needle or catheter configured to enter into the intraarticular space;
(c) a device configured to remove fluid from the intraarticular space;
(d) a source of lavage fluid.

2. The joint lavage kit of claim 1, wherein the source of lavage fluid comprises one or more of a syringe, a bag, tubing, a bottle, or a pump.

3. The joint lavage kit of claim 1, wherein the lavage fluid comprises one or more of: saline, hypertonic fluid, hypotonic fluid, electrolyte solution, anti-inflammatory agent, corticosteroid, hyaluronate, hyaluronate derivative, albumin, plasma, platelet-rich plasma, stem cells, blood-derived products, pr medication.

4. The joint lavage kit of claim 1, wherein the lavage fluid comprises one or more of: normal saline, lactated Ringer's solution, glucose solutions, hypertonic fluid, hypotonic fluid, electrolyte solution, hyaluronate, hyaluronate derivative, albumin, plasma, platelet-rich plasma, or blood-derived products.

5. The joint lavage kit of claim 1, wherein the external compressive device comprises one or more of: a pneumatic brace, an elastomeric brace, a composite brace, or a brace with straps configured to be reversibly engaged to compress and increase pressure on articular soft tissues and reversibly disengaged to decrease pressure on articular soft tissues.

6. The joint lavage kit of claim 1, wherein the needle or catheter is a conventional metal hypodermic needle, or a metal hypodermic needle with fenestrations that prevent clogging and permit greater fluid flow.

7. The joint lavage kit of claim 1, wherein the needle or catheter is a plastic catheter configured to be introduced over or within an introducer metal needle.

8. The joint lavage kit of claim 7, wherein the plastic catheter is configured to curl within the intraarticular space after the introducer needle is removed.

9. The joint lavage kit of claim 7, wherein the plastic catheter has fenestrations that that prevent clogging and permit greater fluid flow.

10. The joint lavage kit of claim 1, wherein the device configured to remove fluid from the intraarticular space comprises one or more of a syringe, reciprocating syringe, refilling syringe, pump, vacuum bottle, or bag.

11. The joint lavage kit of claim 1, further comprising a device to inject lavage fluid into the intraarticular space.

12. The joint lavage kit of claim 11, wherein the device to inject lavage fluid into the intraarticular space comprises one or more of a syringe, reciprocating syringe, refilling syringe, pump, vacuum bottle, or bag.

13. The joint lavage kit of claim 1, further comprising valves, stopcocks, check valves, or combinations thereof mounted between any of the various elements.

14. The joint lavage kit of claim 1, further comprising tubing mounted between the various elements.

15. The joint lavage kit of claim 1, further comprising depth markers comprising metal or plastic configured to mount with the needle or catheter and maintain the needle or catheter at a fixed position in the intraarticular space.

16. The joint lavage kit of claim 1, further comprising a sterile drape.

17. The joint lavage kit of claim 1, further comprising an antiseptic device such as a povidone or chlorhexidine dispenser, pad, or module.

18. The joint lavage kit of claim 1, further comprising gauze for compression and cleaning.

19. The joint lavage kit of claim 1, further comprising at least one bandage adhesive bandage strip to cover the puncture site.

20. The joint lavage kit of claim 1, further comprising a valve controlling fluid communication among the elements.

21. The joint lavage kit of claim 1, wherein the valve comprises a combination of two-, three-, or four-way stopcock, or contains check valve components that enable multiple cycles of fluid exchange to be performed without detaching syringes or other devices.

22. The joint lavage kit of claim 1, further comprising a separate needle and syringe for anesthesia of the skin and tissue prior to introduction of the intraarticular needle or catheter.

23. The joint lavage kit of claim 1, wherein the lavage fluid contains an exogenous marker suitable for determining the dilution of the lavage aspirate relative to the original volume of fluid in the body space treated

24. The joint lavage kit of claim 1, further comprising a collection vessel for lavage aspirate, wherein the collection vessel contains preservatives or protease or both; nuclease, glycosidase and lipase inhibitors; or a combination thereof; to preserve biomarkers for analysis.

25. The joint lavage kit of claim 1, further comprising an analysis kit for biomarkers indicative of arthritis phenotype.

26. A method of performing lavage on a joint, comprising
(a) providing a joint lavage kit as in claim 1;
(b) using the external compression device to move fluid from the articular soft tissues such that it pools at a site that enables easy needle or catheter insertion and fluid extraction;
(c) introducing the needle or catheter into the intraarticular space;
(d) using the device to remove fluid from the intraarticular space.

27. The method of claim 26, wherein the joint lavage kit further comprises a device to introduce lavage fluid into the joint, and further comprising (e) using said device to introduce lavage fluid into the joint after step (d).

28. The method of claim 27, further comprising (f) removing the needle or catheter after step (e).

29. The method of claim 27, further comprising, after step (e), (f) using the external compression device to increase pressure on articular soft tissues, and (g) using said device to remove from the joint at least some of the lavage fluid introduced into the joint in step (e).

30. The method of claim 29, further comprising, after step (g), repeating steps (a) through (g) one or more times.

31. The method of claim 30, further comprising, after the final repeat of step (g), (h) removing the needle or catheter.

31. The method of claim 30, further comprising, after the final repeat of step (g), (h) injecting an intraarticular therapy into the joint, and (i) removing the needle or catheter.

32. The method of claim 27, wherein the lavage fluid comprises one or more of saline, hypertonic fluid, hypotonic fluid, electrolyte solution, anti-inflammatory agent, corticosteroid, hyaluronate, hyaluronate derivative, albumin, plasma, platelet-rich plasma, stem cells, blood-derived products, or medication.

33. The method of claim 27, wherein the lavage fluid comprises one or more of normal saline, lactated Ringer's solution, glucose solutions, hypertonic fluid, hypotonic fluid, electrolyte solution, hyaluronate, hyaluronate derivative, albumin, plasma, platelet-rich plasma, or blood-derived products.

34. The method of claim 27, further comprising fixing the needle or catheter to the skin with adhesive tape or with a depth marker such that the lavage needle is maintained at a fixed position in the intraarticular space.

35. The method of claim 27, wherein step (s) comprises introducing lavage fluid into the synovial membrane.

36. The method of claim 27, wherein step (s) comprises introducing lavage fluid into the intraarticular space.

37. The method of claim 26, wherein the needle or catheter is introduced with the assistance of sonographic, fluoroscopic, radiographic, magnetic resonance imaging, or computed tomographic, guidance, such that the tissue space being dilated can be directly visualized to ensure accurate placement of the needle, complete removal of the aspirate and fluid dissection of adhesions where required.

38. The method of claim 26, further comprising collecting cells from the intraarticular space.
